(19) United States
(12) Patent Application Publication
(10) Pub. No.: US 2013/0053955 A1
(43) Pub. Date: Feb. 28, 2013

(54) INTRAOCULAR LENS (IOL)

(71) Applicant: Gene Currie, Irvine, CA (US)

(72) Inventor: Gene Currie, Irvine, CA (US)

(21) Appl. No.: 13/471,418

(22) Filed: May 14, 2012

Related U.S. Application Data
(63) Continuation-in-part of application No. 11/312,553, filed on Dec. 21, 2005, now Pat. No. 8,216,308.
Application No. 11/312,553 is a continuation-in-part of application No. 11/249,358, filed on Oct. 14, 2005, now abandoned.
Application No. 11/249,358 is a continuation-in-part of application No. 10/942,992, filed on Sep. 17, 2004, now Pat. No. 7,435,258.

(51) Int. Cl. A61F 2/16

(52) U.S. Cl. 623/6.17; 623/6.11; 623/6.43; 623/6.31

(57) ABSTRACT

An improved intraocular lens, for example, an accommodating intraocular lens including a lens optic or lens optic portion provided with a light window.

### RELATED APPLICATIONS

This is a Continuation-In-Part (CIP) of U.S. patent application entitled “Accommodating Artificial Ocular Lens (AAOL) Device”, application Ser. No. 11/312,553 filed on Dec. 21, 2005, now pending, which is a Continuation-In-Part (CIP) of U.S. patent application entitled “Refractive Corrective Lens (RCL)”, application Ser. No. 11/249,358 filed on Oct. 14, 2005, now abandoned, and U.S. patent application entitled “An Accommodating Intraocular Lens Device”, application Ser. No. 10/942,992 filed on Sep. 17, 2004, now U.S. Pat. No. 7,435,258, and all three (3) applications incorporated by reference herein.

### FIELD

An intraocular lens (IOL) device, for example, an accommodating intraocular lens (AIOL) device providing accommodation of vision of the eye.

### BACKGROUND

Currently, there exists a high level of cataract lens surgeries performed in the United States and in other countries and territories throughout the world. These cataract lens surgeries involve the removal of the natural lens, typically by phacoemulsification, followed by the implantation of an aphakic intraocular lens (AIOL). Further, more recently phakic intraocular lens (PIOL), for example, the implantable contact lens (ICL) are implanted with the natural lens still intact.

Most lens surgeries are performed using an intraocular lens providing little if any accommodation of the eye. Specifically, the intraocular lens is implanted into the eye, and once healing of the eye has occurred, there is very little movement of the intraocular lens in a manner to focus the eye by accommodation like the natural crystalline lens of the eye.

There has been much interest in creating and designing intraocular lens configured to provide accommodation the same or similar to the natural crystalline lens of the eye, or provide accommodation in an alternative manner. It is believed that the next generation of intraocular lenses will be accommodating intraocular lenses that provide a significant amount of accommodation of at least one (1) diopter or more. So far, most accommodating intraocular lenses being clinically studied provide one (1) diopter or less of accommodation of the eye. Thus, there now exists a need for an accommodating intraocular lens that can provide a substantial amount of accommodation of the eye, desirably, providing one (1) or more diopters of accommodation of the eye.

The subject matter is directed to an intraocular lens (IOL), for example, an accommodating intraocular lens (AIOL) configured to provide for substantial accommodation of the eye once implanted.

### SUMMARY

A first object is to provide an improved intraocular lens.

A second object is to provide an improved accommodating intraocular lens.

A third object is to provide an intraocular lens comprising or consisting of a lens optic window.

A fourth object is to provide an intraocular lens comprising or consisting of a lens optic configured to provide accommodation of vision of the eye.

A fifth object is to provide an intraocular lens comprising or consisting of a lens optic or lens optic portion comprising a lens optic window configured to provide accommodation of vision of the eye.

A six object is to provide an intraocular lens comprising or consisting of a lens optic or lens optic portion comprising a lens optic window portion configured to provide accommodation of vision of the eye.

A seventh object is to provide an intraocular lens comprising or consisting of a lens optic or lens optic portion comprising a centered lens optic window portion configured to provide accommodation of vision of the eye.

An eighth object is to provide an intraocular lens comprising or consisting of a lens optic or lens optic portion comprising a lens optic window, the lens optic window comprising a flat lens optic portion to provide accommodation of vision of the eye.

A ninth object is to provide an intraocular lens comprising or consisting of a lens optic or lens optic portion comprising a lens optic window, the lens optic window comprising a flat lens surface optic portion to provide accommodation of vision of the eye.

A tenth object is to provide an intraocular lens comprising or consisting of a lens optic or lens optic portion comprising a lens optic window, the lens optic window comprising zero or near zero refraction of light.

A eleventh object is to provide an intraocular lens comprising or consisting of a lens optic or lens optic portion comprising a lens optic window, the lens optic window comprising a positive lens surface on one side and a negative lens surface on an opposite side.

A twelfth object is to provide an intraocular lens comprising or consisting of a lens optic or lens optic portion comprising a lens optic window, the lens optic window comprising a positive lens surface portion on one side and a negative lens surface portion on an opposite side to zero out the power of the combined lens set.

A thirteenth object is to provide an intraocular lens comprising or consisting of a lens optic or lens optic portion comprising a lens optic window, the lens optic window comprising a flat lens optic portion to provide accommodation of vision of the eye.

A fourteenth object is to provide an intraocular lens comprising or consisting of a lens optic or lens optic portion comprising a lens optic window configured as a center flat circular lens optic portion to provide accommodation of vision of the eye.

A fifteenth object is to provide an intraocular lens comprising or consisting of a lens optic or lens optic portion comprising a lens optic window, the lens optic window comprising a center flat non-circular lens optic portion to provide accommodation of vision of the eye.

A sixteenth object is to provide an intraocular lens comprising or consisting of a lens optic or lens optic portion comprising a lens optic window, the lens optic window comprising a diffractive lens.

A seventeenth object is to provide an intraocular lens comprising or consisting of a lens optic or lens optic portion comprising a lens optic window, the lens optic window comprising a hole or recess to provide accommodation of vision of the eye.

An eighteenth object is to provide an intraocular lens comprising or consisting of a lens optic or lens optic portion comprising an optical window, the optical window comprising a lens optic through hole to provide accommodation of vision of the eye.

A nineteenth object is to provide an intraocular lens comprising or consisting of lens optic or lens optic portion including an optical window, the optical window comprising a center lens optic light tunnel to provide accommodation of vision of the eye.

A twentieth object is to provide an intraocular lens comprising or consisting of a lens optic portion including a lens optic window, the lens optic window comprising a non-circular lens optic hole or through hole configured to provide accommodation of vision of the eye.

A twenty first object is to provide an intraocular lens comprising or consisting of a lens optic or lens optic portion comprising a lens optic window, the lens optic window configured as a shaped lens optic hole or through hole to provide accommodation of vision of the eye.

A twenty second object is to provide an intraocular lens comprising or consisting of a lens optic portion and a lens haptic portion, the lens optic portion comprising a lens optic window configured to provide accommodation of vision of the eye.

A twenty third object is to provide an intraocular lens configured to provide static accommodation of vision of the eye.

A twenty fourth object is to provide an intraocular lens configured to provide both static and dynamic accommodation of vision of the eye.

A twenty fifth object of the present invention is to provide an intraocular lens comprising or consisting of a lens optic or lens optic portion comprising a lens optic window configured to provide static accommodation of vision of the eye.

A twenty sixth object is to provide an intraocular lens comprising or consisting of a lens optic portion and a lens haptic portion configured to provide movement between the lens optic portion and lens haptic portion while being implanted in the eye to provide dynamic accommodating of vision of the eye, the lens optic portion including a lens optic window configured to provide static accommodation of the vision of the eye.

A twenty seventh object is to provide an intraocular lens comprising or consisting of a lens optic or lens optic portion comprising an optical window configured as a lens optic insert.

A twenty eighth object is to provide an intraocular lens comprising or consisting of a lens optic or lens optic portion comprising an optical window configured as a lens optic insert configured to provide accommodation of vision of the eye.

A twenty ninth object is to provide an intraocular lens comprising or consisting of a lens optic or lens optic portion comprising an optical tunnel.

A thirtieth object is to provide an intraocular lens comprising or consisting of a lens optic or lens optic portion comprising an optical tunnel configured to provide accommodation of vision of the eye.

A thirty first object is to provide an intraocular lens comprising or consisting of a lens optic or lens optic portion comprising a wave guide.

A thirty second object is to provide an intraocular lens comprising or consisting of a lens optic or lens optic portion comprising a wave guide configured to provide accommodation of vision of the eye.

A thirty third object is to provide an intraocular lens comprising or consisting of a lens optic or lens optic portion comprising an optical fiber.

A thirty fourth object is to provide an intraocular lens comprising or consisting of a lens optic or lens optic portion comprising an optical fiber configured to provide accommodation of vision of the eye.

A thirty fifth object is to provide an intraocular lens comprising or consisting of a lens optic or lens optic portion including a light guide.

A thirty sixth object is to provide an intraocular lens comprising or consisting of a lens optic or lens optic portion including a light guide configured to provide accommodation of vision of the eye.

The subject matter is directed to an improved intraocular lens, for example, an accommodating intraocular lens.

The intraocular lens comprises or consists of a lens optic or lens optic portion. The lens optic can exist by itself, or can be connected to a haptic portion, for example, one or more loop haptic portions or one or more plate haptics portions. The lens optic portion can be hard (e.g. made of hard material, hard plastic, polymethylmethacrylate (PMMA), acrylic, glass, or other biological compatible hard material), or can be deformable or resilient (e.g. made of soft polymer, hydrogel, silicon, collagen based polymer, or other suitable biological compatible materials of suitable refractive index.

The loop haptic, for example, is made of polyester, aramid fiber, or other suitable biocompatible material. The plate haptic portion is preferably made of a substantially flexible or resilient material and configured to allow the plate haptic to deform, flex, or bow when forces and/or pressures are applied by the eye onto the intraocular lens.

The intraocular lens or accommodating intraocular lens can be made as a single piece lens (e.g. by molding or machining), or can be made as a multiple-piece lens assembled together (e.g. hard or soft lens optic portion and resilient or deformable lens haptic portion).

The lens optic or lens optic portion of the intraocular lens or accommodating intraocular lens device can remain stable (e.g. substantially fixed in shape and size after implantation). Specifically, the lens optic or lens optic portion can remain in the same conformation, and is not deformed, bent, or bowed once implanted in the eye. Otherwise, the lens optic portion may not provide consistent light transmission and/or refraction during use or while the accommodating intraocular lens device is performing accommodation within the eye. However, it is to be noted that the lens optic or lens optic portion, in particular for a deformable type intraocular lens or accommodating intraocular lens, can be substantially deformed, rolled or folded during insertion, for example, through a small incision for implantation of the lens device into the eye. This type of lens then regains its operational shape and size, unrolls, or unfolds within the eye for placement in the appropriate position within the eye.

The intraocular lens or accommodating intraocular lens in one embodiment comprises or consists of a lens optic window, in particular a lens optic window configured to provide accommodation of vision of the eye. The lens optic window, for example, is located in the lens optic or lens optic portion of the intraocular lens. Once implanted, this type of intraocular lens provides for “static” accommodation of vision of the eye, since the intraocular lens itself, or parts or portions of the intraocular lens do not have to move relative to each other when implanted in the eye to still provide accommodation of vision of the eye.

The lens optic window (light window) is configured to allow for most light (i.e. light rays) to pass through (e.g. pass straight through) the lens optic window with a minimal to none light refraction or distortion.

It is understood that the intraocular lens may still move somewhat in the eye once implanted. For example, an aphakic intraocular lens may move when implanted into the capsular bag due to movement of the capsular bag, and a phakic intraocular lens may move with movement of the natural crystalline lens of the eye. Further, an anterior chamber lens may move very little or not at all. However, accommodation of vision of the eye is still provided based on optical principles even when the intraocular lens moves slightly or not at all when implanted in the eye.

The lens optic window can be provided in various arrangements or embodiments. For example, in one embodiment the lens optic window comprises a flat lens optic portion provided on one or both sides of the lens optic or lens optic portion. Preferably, a pair of overlapping flat lens optic portions (e.g. flat surfaces) are provided on each side of the lens optic or lens optic portion. The flat lens optic portions can, for example, be flat circular lens optic portions centered and overlapping and of the same size and shape provided on both sides of the lens optic or lens optic portion.

The flat lens optic portion can be provided on one or both sides of the lens optic portion, and can overlap, partially overlap, or not overlap. Further, the shape and size of the flat lens optic portion can vary on one or both sides of the lens optic or lens optic portion. For example, the flat lens optic portion is a flat shaped lens optic portion (e.g. circular-shaped or round, oval, triangle, square, pentagon, hexagon, octagon, star-shaped, gear-shaped).

The flat lens optic portion is configured to allow light to pass more directly through (e.g. straight through) the thickness of the lens optic or lens optic portion (e.g. pass through lens optic or lens optic portion perpendicular relative to a flat surface of the lens optic portion).

The flat lens optic portion(s), for example, can dimensionally be in the range of 0.5 mm to 4 mm, can be in the range of 0.5 mm to 3 mm, can be in the range of 0.5 mm to 2 mm, or can be approximately 1 mm. Further, the flat lens optic portion(s) can be circular and have a diameter(s) in the range of 0.1 mm to 4 mm, can be in the range of 0.5 mm to 3 mm, can be in the range of 0.5 mm to 2 mm, or can be approximately 1 mm.

In another embodiment, the lens optic window comprises a lens optic tunnel (e.g. hole or holes, partial depth holes or recesses, tunnel, through hole, overlapping partial holes or recesses on both sides). Preferably, the lens optic tunnel can be circular-shaped or round, and centered in the lens optic or lens optic portion. The lens optic light tunnel can be provided on one or both sides of the lens optic or lens optic portion, and can overlap, partially overlap, or not overlap. Further, the shape and size of the lens optic light tunnel can vary on one or both sides of the lens optic or lens optic portion. For example, the lens optic light tunnel is specially shaped in two or three dimensions (e.g. circular-shaped or round, oval, triangle, square, pentagon, hexagon, octagon, star-shaped, gear-shaped, cylindrical-shaped, cone-shaped, tapering, funnel shaped, prism shaped).

The lens optic light tunnel is configured to allow light to pass more directly, or directly through the thickness of the lens optic or lens optic portion (i.e. pass through lens optic portion along focal axis or perpendicular to lens optic or lens optic portion surface and without passing through reduced thickness or no lens material).

For example, the lens optic light tunnel can have a width in the range of 0.5 mm to 4 mm, can be in the range of 0.5 mm to mm, can be in the range of 0.5 mm to 2 mm, or can be approximately 1 mm. Further, the lens optic light tunnel can be circular and have a diameter in the range of 0.5 mm to 4 mm, can be in the range of 0.5 mm to 3 mm, can be in the range of 0.5 mm to 2 mm, or can be approximately 1 mm.

In a further embodiment, the lens optic window comprises a flat lens optic portion and a lens optic light tunnel. Preferably, the flat lens optic portion and lens optic light tunnel are circular-shaped or round, and centered on the lens optic or lens optic portion. The flat lens optic portion and/or lens optic light tunnel can be provided on one or both sides of the lens portion, and can overlap, partially overlap, or not overlap, or configured as a through hole. Further, the shape and size of the flat lens optic portion and/or lens optic light tunnel can vary on one or both sides of the lens optic or lens optic portion. For example, the flat lens optic portion and/or lens optic light tunnel are shaped (e.g. circular-shaped or round, oval, triangle, square, pentagon, hexagon, octagon, star-shaped, gear-shaped). The combination of the flat lens optic portion and lens optic light tunnel are configured to allow light to pass directly through the thickness of the lens optic or lens optic portion (i.e. pass through lens optic or lens optic portion perpendicular to lens optic or lens optic portion surface and travel the least distance through thickness of the lens optic or lens optic portion).

For example, the flat lens optic portion and/or lens optic light tunnel can dimensionally be in the range of 0.5 mm to 4 mm, can be in the range of 0.5 mm to 3 mm, can be in the range of 0.5 mm to 2 mm, or can be approximately 1 mm. Further, the flat lens optic portion and/or the lens optic light tunnel can be circular and have diameters in the range of 0.5 mm to 4 mm, can be in the range of 0.5 mm to 3 mm, can be in the range of 0.5 mm to 2 mm, or can be approximately 1 mm.

In an even further embodiment, the lens optic window comprises a lens optic insert. The lens optic insert, for example, can be provided in the lens optic or lens optic portion and configured to provide accommodation of vision of the eye.

For example, the lens optic insert can dimensionally be in the range of 0.5 mm to 4 mm, can be in the range of 0.5 mm to 3 mm, can be in the range of 0.5 mm to 2 mm, or can be approximately 1 mm. Further, the lens optic insert can be circular and have diameters in the range of 0.5 mm to 4 mm, can be in the range of 0.5 mm to 3 mm, can be in the range of 0.5 mm to 2 mm, or can be approximately 1 mm.

In another embodiment, the lens optic window comprises a positive lens optic surface on one side of the lens optic and a negative lens optic surface on an opposite side of the lens optic. For example, the positive lens optic surface can cancel out the negative lens optic surface to zero out the lens power.

For example, the lens optic window, or lens optic surfaces, can dimensionally be in the range of 0.5 mm to 4 mm, can be in the range of 0.5 mm to 3 mm, can be in the range of 0.5 mm to 2 mm, or can be approximately 1 mm. Further, the lens optic insert can be circular and have diameters in the range of 0.5 mm to 4 mm, can be in the range of 0.5 mm to 3 mm, can be in the range of 0.5 mm to 2 mm, or can be approximately 1 mm.

In a further embodiment, the lens optic window comprises a diffractive lens optic portion.

For example, the lens optic window, or diffractive lens optic portion, can dimensionally be in the range of 0.5 mm to 4 mm, can be in the range of 0.5 mm to 3 mm, can be in the range of 0.5 mm to 2 mm, or can be approximately 1 mm. Further, the lens optic insert can be circular and have diameters in the range of 0.5 mm to 4 mm, can be in the range of 0.5 mm to 3 mm, can be in the range of 0.5 mm to 2 mm, or can be approximately 1 mm.

The intraocular lens or accommodating intraocular lens in an even further embodiment comprises or consists of a lens optic portion and a haptic portion. The lens optic portion can be connected to the lens haptic portion.

The intraocular lens or accommodating intraocular lens can comprise or consist of a movable lens optic portion and a fixed lens haptic portion, preferably a plate lens haptic portion, relative to the eye in particular the inner eye structure. The lens optic portion functions essentially as a separate component relative to the lens haptic portion, except for at least one flexible or resilient arm connecting the lens optic portion to the lens haptic portion. Preferably, at least one opening is provided between an outer edge of the lens optic portion and an inner edge of the plate lens haptic portion. More specifically, the lens optic portion is separated from most of the plate lens haptic portion by a space (e.g. fixed or varying distance). In this arrangement, the outer edge of the lens optic portion is able to move substantially freely and independently relative to the inner edge of the plate lens haptic portion, except at the point or points of connection with the flexible or resilient arm(s).

This embodiment provides for a type of accommodation with movement of the lens optic portion relative to the lens haptic portion. This embodiment can be combined with a flat lens optic portion and/or lens optic light tunnel to provide additional accommodation (i.e. accommodation provide by both static and dynamic arrangements to enhance accommodation effect).

In the accommodative movement embodiment, the intraocular lens or accommodating intraocular lens comprises or consists of at least one arm, preferably a flexible or resilient arm connecting the lens optic portion to the plate lens haptic portion. The flexible or resilient arm is configured to move the lens optic portion along the central focus axis of the eye when the plate lens haptic portion is deformed, flexed, and/or bowed, for example, when the eye exerts inwardly and/or outwardly radial forces at one or more positions around the outer edge of the plate lens haptic portion and/or on the front or back surfaces of the lens. More specifically, the lens optic portion is moved along the central focal axis of the eye for purposes of accommodation for focusing the eye when the plate lens haptic portion is bowed. At least one flexible arm allows the outer edge of the lens optic portion to move relative to the inner edge of the plate lens haptic portion, which becomes distorted as the plate lens haptic portion is bowed. In this manner, the at least one flexible or resilient arm accommodates tensile stress, shear stress and some torsional stress when the plate lens haptic portion is deformed, flexed, and/or bowed without breaking or permanently deforming. Specifically, the stresses are at a level within the at least one flexible or resilient arm when the plate lens haptic portion is deformed, flexed, and/or bowed so as to not cause plastic deformation of the at least one flexible or resilient arm. Thus, when the force on the lens is relieved, the stresses within the at least one arm are relieved and causes the lens optic portion to move back to a resting position relative to the eye.

The accommodative movement, for example, of the lens optic portion is initially located in the same plane as the plate lens haptic portion. When force is applied to the outer edge of the plate lens haptic portion, the plate lens haptic portion begins to bow and moves the lens optic portion out of the initial reference plane along the central focal axis of the eye. As the plate lens haptic portion bows, it changes shape from substantially from a two (2) dimensional configuration to a cupped or bowed three (3) dimensional configuration, and is substantially no longer planar.

The outer periphery or edge portion of the plate lens haptic portion can be configured and/or treated to facilitate or enhance anchoring thereof within the eye. Specifically, the plate lens haptic portion can be provided with one or more through holes to allow tissue on either side of the plate lens haptic portion to adhere together in the through hole. Alternatively, or in addition, the outer edge of the lens plate haptic portion can be provided with surface treatment (e.g. porous), scallops, serrations and/or notches to facilitate tissue adherence thereto.

In the accommodating intraocular lens, the lens optic portion can be circular shaped or oval shaped. Further, the lens optic portion can be centered relative to the shape of the outer periphery of the plate lens haptic portion.

As an alternative embodiment, the lens optic portion can be located off centered in one or both the length and width dimensions relative to the outer perimeter of the plate lens haptic portion. In this manner, the accommodating intraocular lens device can be customized to take into account the morphology of the interior structure of the eye of a particular patient in custom designing and prescribing the particular accommodating intraocular lens device for the particular patient.

The accommodating intraocular lens can have a pair of flexible or resilient arms connecting the lens optic portion to the plate lens haptic portion, the lengths of the arm portions can be the same or different. Further, the location of the pair of arm portions can be located along a center axis of the lens optic portion or can be located off axis.

### BRIEF DESCRIPTION OF THE DRAWINGS

FIG. 1 is a perspective view of a deformable accommodating intraocular lens device shown under a stressed condition providing forward accommodation of the lens portion along the center focal axis of the eye.

FIG. 2 is a top planar view of the deformable accommodating intraocular lens device, as shown in FIG. 1.

FIG. 3 is a cross-sectional view of the deformable accommodating intraocular lens device, as indicated in FIG. 2, in an unstressed condition.

FIG. 4 is a cross-sectional view, as shown in FIG. 3, when the lens device is in a stressed condition.

FIG. 5 is a top planar view of another embodiment of the accommodating intraocular lens device.

FIG. 6 is a cross-sectional view of the accommodating intraocular lens device as indicated in FIG. 5.

FIG. 7 is a partial broken away detailed cross-sectional view of a portion of the accommodating intraocular lens device shown in FIGS. 5 and 6.

FIG. 8 is a top planar view of another embodiment of an accommodating intraocular lens.

FIG. 9 is a top planar view of another embodiment of the accommodating intraocular lens.

FIG. 10 is a top planar view of another embodiment of the accommodating intraocular lens.

FIG. 11 is a top planar view of another embodiment of the accommodating intraocular lens.

FIG. 12 is a top planar view of another embodiment of the accommodating intraocular lens.

FIG. 13 is a top planar view of another embodiment of an accommodating intraocular lens.

FIG. 14 is a top planar view of another embodiment of the accommodating intraocular lens.

FIG. 15 is a top planar view of a further embodiment of the accommodating intraocular lens.

FIG. 16 is a top planar view of a further embodiment of the accommodating intraocular lens.

FIG. 17 is a top planar view of a further embodiment of the accommodating intraocular lens.

FIG. 18 is a top planar view of a further embodiment of the accommodating intraocular lens.

FIG. 19 is a top planar view of a further embodiment of the accommodating intraocular lens.

FIG. 20 is a top planar view of a further embodiment of the accommodating intraocular lens.

FIG. 21 is a perspective view of an accommodating intraocular lens device having double lens portions.

FIG. 22 is a center cross-sectional view of the accommodating intraocular lens shown in FIG. 21.

FIG. 23 is a top planar view of the lens shown in FIG. 2 in a resting or unstressed condition.

FIG. 24 is a center cross-sectional view of the accommodating intraocular lens, as indicated in FIG. 23.

FIG. 25 is a center cross-sectional view of the accommodating intraocular lens shown in FIG. 24 in a stressed condition showing the plate lens haptic portion bowing and the lens optic portion moving along the optical axis Z of the eye.

FIG. 26 is a center cross-sectional view of the accommodating intraocular lens as indicated in FIG. 23, along the transverse axis Y.

FIG. 27 is a perspective view of an embodiment of the accommodating intraocular lens comprising or consisting of a lens optic window comprising a flat lens optic portion provided in the lens optic portion.

FIG. 28 is a top planar view of the accommodating intraocular lens shown in FIG. 27.

FIG. 29 is a side elevational of the accommodating intraocular lens shown in FIG. 27.

FIG. 30 is a top planer view of an embodiment of the accommodating intraocular lens comprising or consisting of a lens optic window comprising a raised flat lens optic portion provided in the lens optic portion.

FIG. 31 is a side elevational view of the accommodating intraocular lens shown in FIG. 30.

FIG. 32 is a top planar view of an embodiment of the accommodating intraocular lens comprising or consisting of a lens optic window comprising a hole provided in the lens optic portion.

FIG. 33 is a side elevational view of the accommodating intraocular lens shown in FIG. 32.

FIG. 34 is a top planar view of an accommodating intraocular lens comprising or consisting of a lens optic window comprising a tapering hole provided in the lens optic portion.

FIG. 35 is a side elevational view of the accommodating intraocular lens shown in FIG. 34.

FIG. 36 is a center cross-sectional view of an accommodating intraocular lens comprising or consisting of a lens optic window comprising a flat lens optic portion and a lens optic hole provided in the lens optic portion.

FIG. 37 is a center cross-sectional view of an accommodating intraocular lens comprising or consisting of a lens optic window comprising a raised flat lens optic portion provided in the lens optic portion.

FIG. 38 is a center cross-sectional view of an accommodating intraocular lens comprising or consisting of a lens optic window comprising a lens optic hole provided in the lens optic portion.

FIG. 39 is a center cross-sectional view of an accommodating intraocular lens comprising or consisting of a lens optic window comprising a tapering lens optic hole provided in the lens optic portion.

FIG. 40 is a center cross-sectional view of an accommodating intraocular lens comprising or consisting of a lens optic window comprising a partial lens optic hole provided in the lens optic portion.

FIG. 41 is a center cross-sectional view of an accommodating intraocular lens comprising or consisting of a lens optic window comprising a closed sided lens optic hole or cavity provided in the lens optic portion.

FIG. 42 is a center cross-sectional view of an accommodating intraocular lens comprising or consisting of a lens optic window comprising a pair of partial tapering holes provided in the lens optic portion.

FIG. 43 is a center cross-sectional view of an accommodating intraocular lens comprising or consisting of a lens optic window comprising a fiber optic lens optic insert provided in the lens optic portion.

FIG. 44 is a center cross-sectional view of an accommodating intraocular lens comprising or consisting of a lens optic window comprising a partial hole or recess provided in the lens optic portion.

FIG. 45 is a center cross-sectional view of an accommodating intraocular lens comprising or consisting of a lens optic window comprising a partial outwardly tapering hole provided in the lens optic portion.

FIG. 46 is a center cross-sectional view of an accommodating intraocular lens comprising or consisting of a lens optic window comprising a pair of partial outwardly tapering holes provided in the lens optic portion.

FIG. 47 is a center cross-sectional view of an accommodating intraocular lens comprising or consisting of a lens optic window comprising a partial inwardly tapering hole raised flat lens optic portion provided in the lens optic portion.

FIG. 48 is a center cross-sectional view of an accommodating intraocular lens comprising or consisting of a lens optic window comprising a tapering lens optic insert provided in the lens optic portion.

FIG. 49 is a center cross-sectional view of an accommodating intraocular lens comprising or consisting of a lens optic window comprising a pair of outwardly tapering lens optic inserts provided in the lens optic portion.

FIG. 50 is a center cross-sectional view of an accommodating intraocular lens comprising or consisting of a lens optic window comprising an outwardly tapering lens optic insert provided in the lens optic portion.

FIG. 51 is a center cross-sectional view of an accommodating intraocular lens comprising or consisting of a lens optic window comprising a bundled fiber optic lens optic insert provided in the lens optic portion.

FIG. 52 is a center cross-sectional view of an accommodating intraocular lens comprising or consisting of a lens optic window comprising a positive lens optic surface portion and a negative lens optic surface portion.

FIG. 53 is a center cross-sectional view of an accommodating intraocular lens comprising or consisting of a lens optic window comprising a negative lens optic surface portion and a positive lens optic surface portion.

FIG. 54 is a center cross-sectional view of an accommodating intraocular lens comprising or consisting of a lens optic window comprising a diffractive lens portion.

### DETAILED DESCRIPTION OF PREFERRED EMBODIMENTS

A deformable accommodating intraocular lens device 10 is shown in FIGS. 1 to 4. This embodiment provides both dynamic accommodation and static accommodation of the vision of the eye.

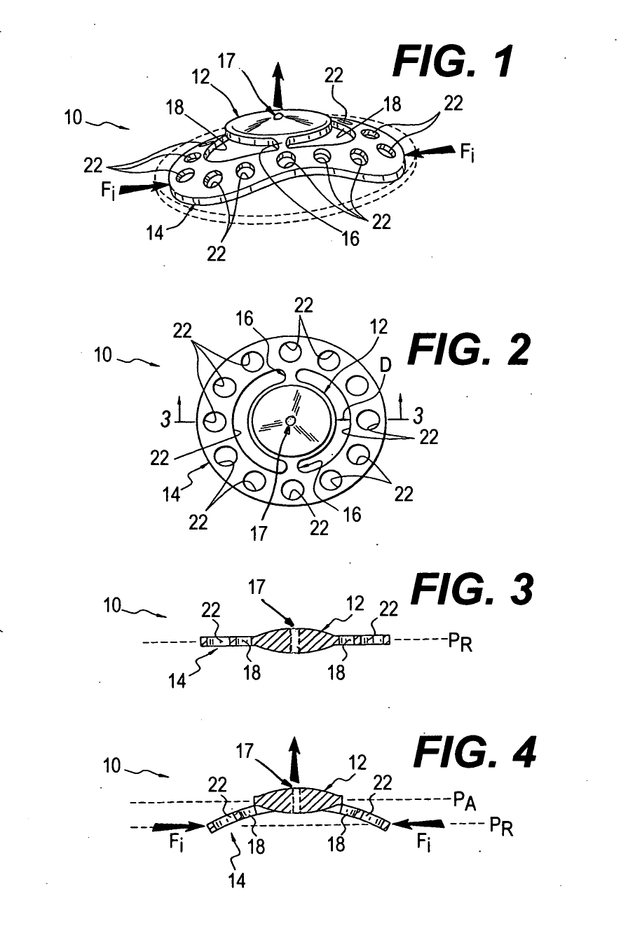

The deformable accommodating intraocular lens device 10 comprises or consists of a lens optic portion 12 and a plate lens haptic portion 14. The lens optic portion 12 is connected to the plate lens haptic portion 14 by a pair of flexible or resilient arm portions 16, 16, as shown in FIG. 2. The lens optic portion 12 is provided with a lens optic window 17.

A pair of partial circular-shaped openings 18, 18 separate the lens optic portion 12 from the plate lens haptic portion 14, as shown in FIG. 2, by a predetermined distance D. In this manner, the lens optic portion 12 is structurally substantially independent of plate lens haptic portion 18, except at the two (2) points of connection provided by the resilient or flexible arm portion 16, 16.

The perimeter of the plate lens haptic portion 14 is provided with a plurality of through holes 22 to facilitate adherence of tissue looped through the through holes 22 by tissue located on either side of the perimeter of the plate lens haptic portion 14 connecting together in the through holes 22. In this manner, once the deformable accommodating intraocular lens device 10 has been implanted and the eye has healed, the perimeter of the plate lens haptic portion 14 becomes substantially anchored in place.

In the embodiments shown in FIGS. 1 to 4, the lens portion 14, plate lens haptic portion 14, and arm portions 16, 16 are made as a one-piece unitary structure from soft, deformable or resilient polymer material. The deformable accommodating intraocular lens device 10 can be inserted through a small incision (e.g. less than 2.5 millimeters) through the cornea of the eye in a deformed rolled, folded or otherwise compressed condition by use of forceps or a lens injecting device.

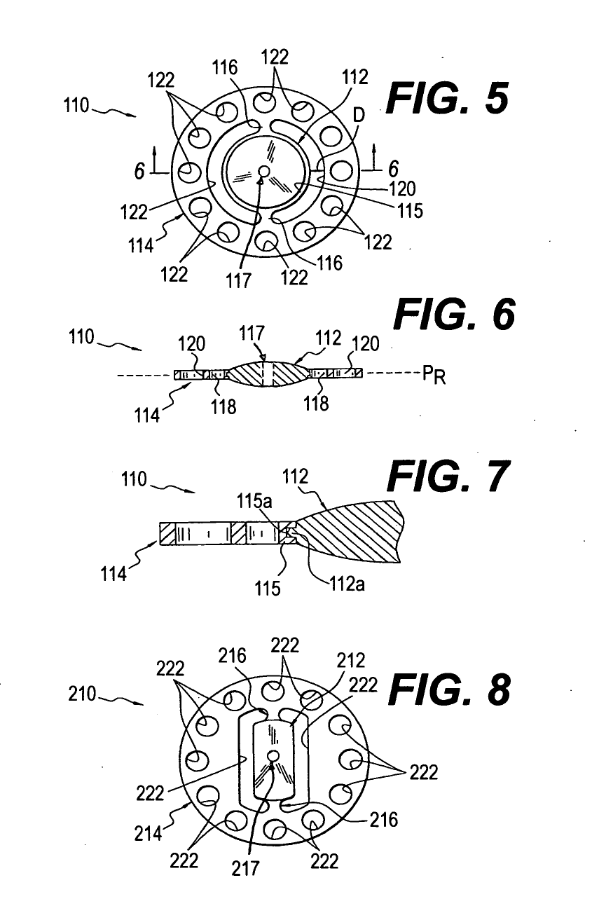

Another embodiment of a partially deformable accommodating intraocular lens 110 device is shown in FIGS. 5-7.

The partially deformable accommodating intraocular lens device 110 comprises or consists of a lens optic portion 112 and a plate lens haptic portion 114. The lens optic portion 112 is connected to the plate lens haptic portion 114 by a pair of resilient or flexible arm portions 116. The lens optic portion 112 is provided with a lens optic window 117.

In this embodiment, the lens optic portion 112 is made out of non-resilient or non-deformable material such as polymethyl methacrylate. However, the plate lens haptic portion 114 is made from a resilient polymer material and the partially deformable accommodating intraocular lens 110 is made from two (2) separate pieces and assembled together to become a single piece lens. Further, the deformable accommodating lens can be made of a material that varies in hardness or stiffness along its length (e.g. harder lens portion and softer plate haptic portions or reverse).

The plate lens haptic portion 114 includes a resilient lens optic carrier or lens optic receiving portion 115 provided with an inner groove 115a cooperating with a tongue portion 112a of the lens optic portions 112 as shown in FIG. 7. The lens optic portion 112 can be secured in place due to the resilient nature of the lens optic receiver or lens optic carrier 115 of the plate lens haptic portion 114 due to its capacity to withstand a certain amount of band stress.

Alternatively, or in addition, the tongue portion 112a can be adhered by glue, adhesive, welding or other technique to secure the lens optic portion 112 to the lens optic receiver or lens optic carrier 115 of the plate lens haptic portion 114.

The partially deformable accommodating intraocular lens device is inserted through a relatively large incision in the cornea by forceps and then implanted into the capsular bag after cataract lens removal.

A further embodiment of the accommodating intraocular lens 210 is shown in FIG. 8.

The accommodating intraocular lens 210 comprises or consists of a substantially rectangular lens optic portion 212 connected to a round-shaped plate lens haptic portion 214 by a pair of flexible or resilient arm portions 216, 216. The lens optic portion 212 is provided with a lens optic window 217.

A pair of oblong or partial oval-shaped openings 220, 220 are provided between the lens optic portion 212 and the plate lens haptic portion 214. A plurality of through holes 222 are provided to facilitate anchoring of the perimeter of the plate lens haptic portion 214.

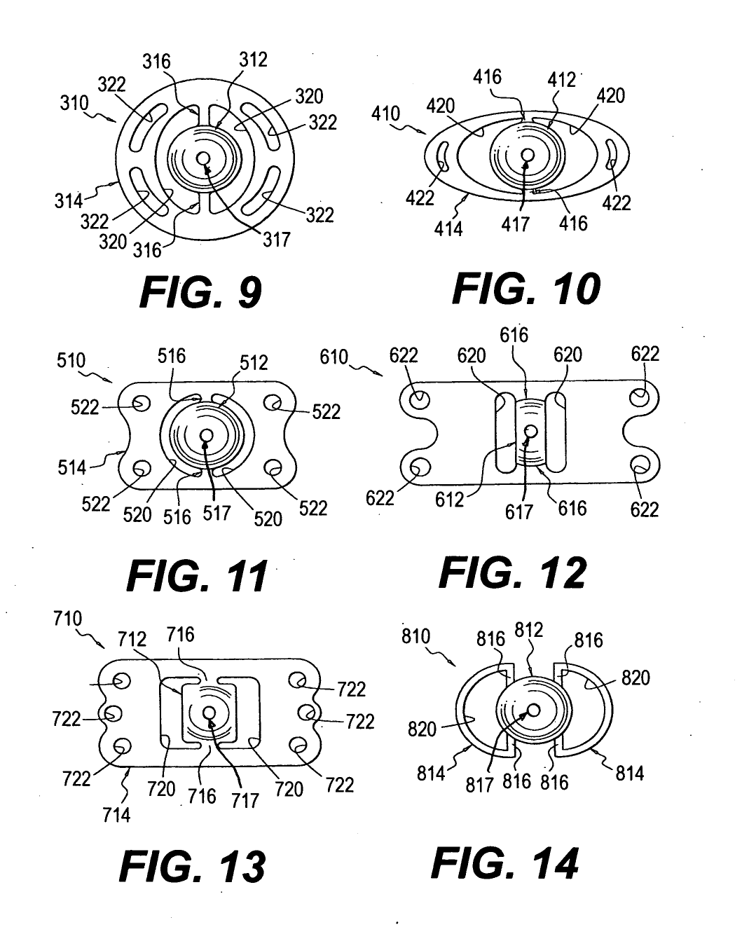

A further embodiment of the accommodating intraocular lens 310 is shown in FIG. 9.

The accommodating intraocular lens 310 comprises or consists of a substantially round lens optic portion 312 connected to a round plate lens haptic portion 314 by a pair of flexible or resilient arm portions 316, 316. The lens optic portion 312 is provided with a lens optic window 317.

A pair of oblong or partial oval-shaped openings 320, 320 are provided between the lens optic portion 312 and the plate lens haptic portion 314. A plurality of through holes 322 are provided to facilitate anchoring of the perimeter of the plate lens haptic portion 314.

A further embodiment of the accommodating intraocular lens 410 is shown in FIG. 10.

The accommodating intraocular lens 410 comprises or consists of a substantially round lens optic portion 412 connected to an oblong-shape plate lens haptic portion 414 by a pair of flexible or resilient arm portions 416, 416. The lens optic portion 412 is provided with a lens optic window 417.

A pair of oblong or partial oval-shaped openings 420, 420 are provided between the lens optic portion 412 and the plate lens haptic portion 414. A plurality of through holes 422 are provided to facilitate anchoring of the perimeter of the plate lens haptic portion 414.

A further embodiment of the accommodating intraocular lens 510 is shown in FIG. 11.

The accommodating intraocular lens 510 comprises or consists of a round lens optic portion 512 connected to a modified oblong-shape plate lens haptic portion 514 by a pair of flexible or resilient arm portions 516, 516. The lens optic portion 512 is provided with a lens optic window 517.

A pair of oblong or partial oval-shaped openings 520, 520 are provided between the lens optic portion 512 and the plate lens haptic portion 514. A plurality of through holes 522 are provided to facilitate anchoring of the perimeter of the plate lens haptic portion 514.

Another embodiment of the accommodating intraocular lens 610 is shown in FIG. 12.

The accommodating intraocular lens 610 comprises or consists of an elongated lens optic portion 612 connected to an elongated plate lens haptic portion 614 by a pair of flexible or resilient arm portions 616, 616. The lens optic portion 612 is provided with a lens optic window 617.

A pair of oblong-shaped openings 620, 620 are provided between the lens optic portion 612 and the plate lens haptic portion 614. A plurality of through holes 622 are provided to facilitate anchoring of the ends of the plate lens haptic portion 614 in the eye.

A further embodiment of the accommodating intraocular lens is shown in FIG. 13. The accommodating intraocular lens 710 comprises or consists of a rectangular lens optic portion 712 connected to a rectangular plate lens haptic portion 714 by a pair of flexible or resilient arm portions 716, 716. The lens optic portion 712 is provided with a lens optic window 717.

A pair of rectangular oval-shaped openings 720, 720 are provided between the lens optic portion 712 and the plate lens haptic portion 714. A plurality of through holes 722 are provided to facilitate anchoring of the ends of the plate lens haptic portion 714 in the eye.

An even further embodiment of the accommodating intraocular lens 810 is shown in FIG. 14.

The accommodating intraocular lens 810 comprises or consists of a round lens optic portion 812 connected to a pair of half-circle plate lens haptic portions 814, 814 each by a pair of flexible or resilient arm portions 816, 816. The lens optic portion 812 is provided with a lens optic window 817.

A pair of half-circle shaped openings 820, 820 are provided between the lens optic portion 812 and the plate lens haptic portions 814, 814. In this embodiment, the openings 820, 820 also provide the function of through holes in previous embodiments to facilitate anchoring the ends of the plate lens haptic portions 814, 814 in the eye.

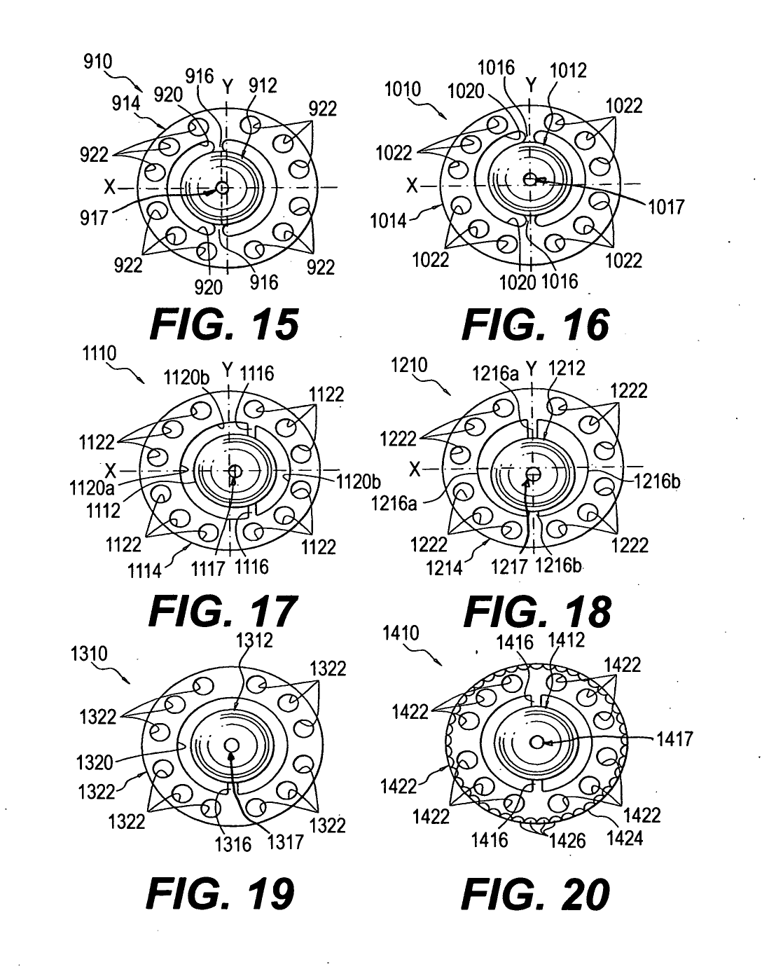

Another embodiment of the accommodating intraocular lens 910 is shown FIG. 15.

The accommodating intraocular lens 910 comprises or consists of a round lens optic portion 912 connected to a round plate lens haptic portion 914 by a pair of flexible or resilient arm portions 916, 916. The arm portions 916, 916 are approximately the same length. The lens optic portion 912 is provided with a lens optic window 917.

A pair of half circular-shaped openings 920, 920 are provided between the lens optic portion 912 and the plate lens haptic portion 914. A plurality of through holes 922 are provided to facilitate anchoring the plate lens haptic portion 914 in the eye. In this embodiment, the lens optic portion 912 is located off centered along the Y axis making the round plate lens haptic portion somewhat asymmetrical in shape relative to the X axis.

Another embodiment of the accommodating intraocular lens 1010 is shown in FIG. 16.

The accommodating intraocular lens 1010 comprises or consists of a round lens optic portion 1012 connected to a round plate lens haptic portion 1014 by a pair of flexible or resilient arm portions 1016, 1016. The lens optic portion 1012 is provided with a lens optic window 1017.

A pair of half circle-shaped openings 1020, 1020 are provided between the lens optic portion 1012 and the plate lens haptic portion 1014. A plurality of through holes 1022 are provided to facilitate anchoring the perimeter of the plate lens haptic portion 1014 in the eye. In this embodiment, the lens portion 1012 is located off center along the X axis resulting in the plate lens haptic portion 1014 being asymmetrical relative to the Y axis.

Another embodiment of the accommodating intraocular lens 1110 is shown in FIG. 17.

The accommodating intraocular lens 1110 comprises or consists of a round lens optic portion 1112 connected to a round plate lens haptic portion 1114 by a pair of flexible or resilient arm portions 1116, 1116. The arm portions 1116, 1116 are both located off axis relative to the Y axis. The lens optic portion 1112 is provided with a lens optic window 1117.

A pair of half circular-shaped openings 1120a, 1120b are provided between the lens optic portion 1112 and the plate lens haptic portion 1114. It is to be noted that the opening 1120a is larger than the opening 1120b. A plurality of through holes 1122 are

provided to facilitate anchoring of the perimeter of the plate lens haptic portion 1114 in the eye.

Another embodiment of the accommodating intraocular lens 1210 is shown in FIG. 18.

The accommodating intraocular lens 1210 comprises or consists of a round lens optic portion 1212 connected to a round plate lens haptic portion 1214 by a pair of flexible or resilient arm portions 1216a and 1216b. It is to be noted that the arm portion 1216a is longer than the arm portion 1216b. The lens optic portion 1212 is provided with a lens optic window 1217.

A pair of asymmetrical half circular-shaped openings 1220 are provided between the lens optic portion 1212 and the plate lens haptic portion 1214. A plurality of through holes 1222 are provided to facilitate anchoring of the perimeter of the plate lens haptic portion 1214 in the eye.

Another embodiment of the accommodating intraocular lens 1310 is shown in FIG. 19.

The accommodating intraocular lens 1310 comprises or consists of a round lens optic portion 1312 connected to a round plate lens haptic portion 1314 by a single flexible or resilient arm portion 1316. The lens optic portion 1312 is provided with a lens optic window 1317.

A single circular-shaped opening 1320 is provided to separate the lens optic portion 1312 from the plate lens haptic portion 1314. A plurality of through holes 1322 are provided to facilitate anchoring of the perimeter of the plate lens haptic portion 1314 in the eye.

Another embodiment of the accommodating intraocular lens 1410 is shown in FIG. 20.

The accommodating intraocular lens 1410 comprises or consists of a round lens optic portion 1412 connected to a round plate lens haptic portion 1414 by a pair of flexible or resilient arm portions 1416, 1416. The lens optic portion 1412 is provided with a lens optic window 1417.

A pair of half circular-shaped openings 1420, 1420 are provided between the lens optic portion 1412 and the plate lens haptic portion 1414. A plurality of through holes 1422 are provided to facilitate anchoring of the perimeter of the plate lens haptic portion 1414 in the eye. In addition, the edge 1424 is provided with scalloped portions 1426 around the perimeter thereof to facilitate the fibrotic fixation process. Alternatively, or in addition, the scallops can be replaced with a roughened surface, porous surface, serrations, notches and/or flaps.

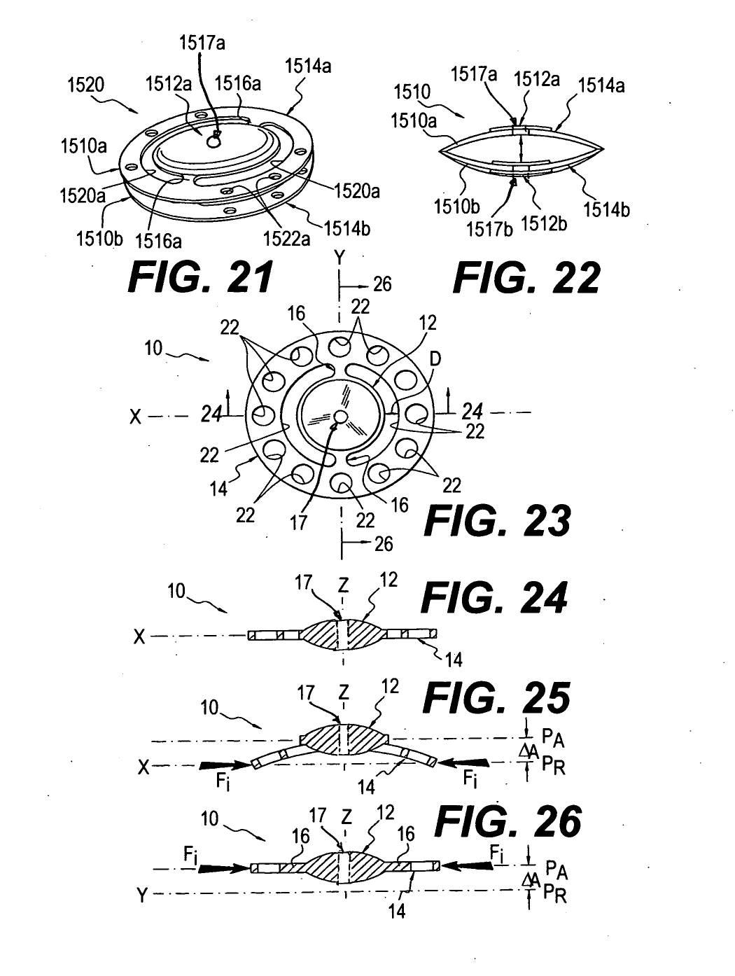

A double lens embodiment of the accommodating intraocular lens 1510 is shown in FIGS. 21 and 22.

The accommodating lens 1510 comprises or consists of a front accommodating lens optic portion 1510a and a back accommodating lens optic portion 1510b. The lens optic portion 1510a and the lens optic portion 1510b are essentially the same configuration except reverse oriented and assembled back-to-back.

The accommodating intraocular lens portion 1510a includes a round lens optic portion 1512a connected to a round plate lens haptic portion 1514a by a pair of flexible or resilient arm portions 1516a, 1516a. The lens optic portions 1512a is provided with a lens optic window 1517a.

A pair of half circular-shaped openings 1520a, 1520a are provided between the lens optic portion 1512a and the plate lens haptic portion 1514a. A plurality of through holes 1522a are provided to facilitate anchoring of the perimeter of the plate lens haptic portion 1514a in the eye.

The accommodating lens portion 1510b is the same or similarly configured to the accommodating lens portion 1510a. The lens optic portion 1512b is provided with a lens optic window 1517b.

As shown in FIG. 22, the outer edges or perimeter of the lens portions 1510a and 1510b contact, engage, or connected to each other when implanted in the eye.

An embodiment of the accommodating intraocular lens haptic portion can have a length preferably from 8 millimeters to 13 millimeters, a width from 5 to 13 millimeters, and a thickness from 0.05 millimeters to 1 millimeter. The opening distance D is preferably 0.20 to 3.0 millimeters. It is important that the ratio of the radial length of the plate lens haptic portion relative to the axial thickness of the plate lens haptic portion is preferably 1.5 to 8 or more, to provide sufficient bowing of the plate lens haptic portion when stressed inwardly by forces applied by the eye.

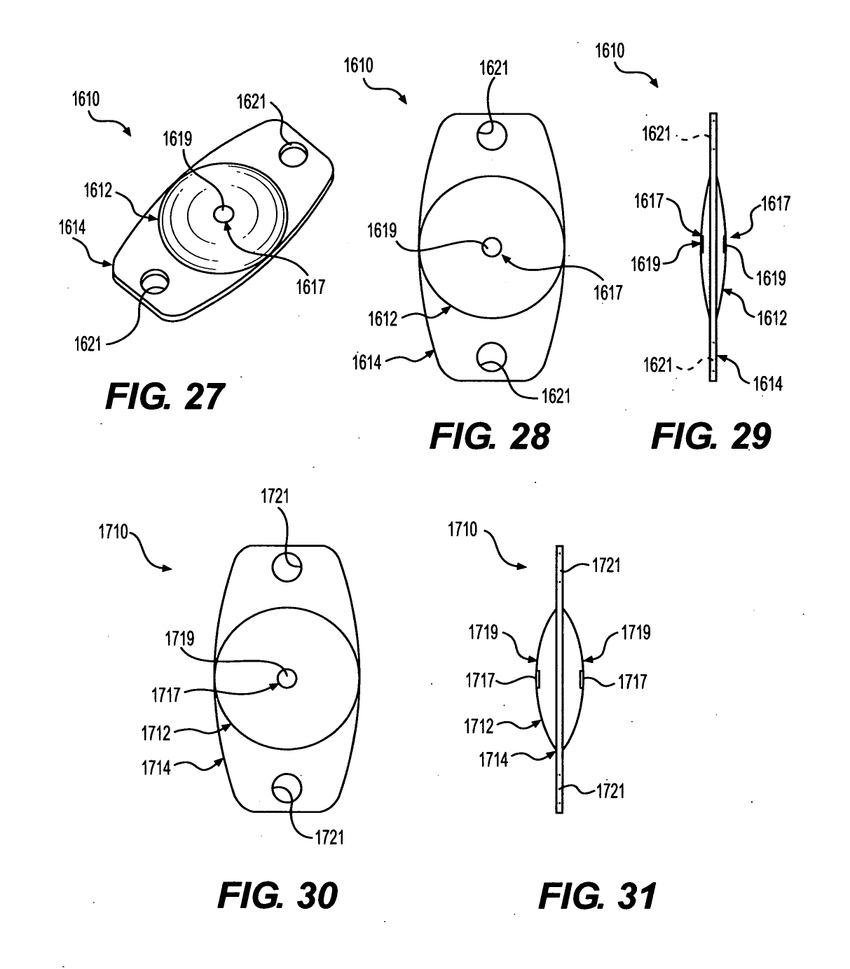

A preferred embodiment of a deformable accommodating intraocular lens device 1610 is shown in FIGS. 27-29. The deformable accommodating intraocular lens 1610 provides for static accommodation of vision of the eye.

The deformable accommodating intraocular lens 1610 comprises or consists of a lens optic portion 1612, a plate lens haptic portion 1614, and a lens optical window 1617. In this embodiment, the lens optical window 1617 is configured as a flat lens optic portion 1619 provided on at least one side of the lens optic portion 1612. Alternatively, the flat lens optic portion 1619 can be located off-center (e.g. slightly off-center).

The flat lens optic portion 1619 is a portion of the lens optic portion 1612 having slight curvature or no curvature relative to the surrounding lens optic portion 1612. Preferably, the flat lens optic portion 1619 is made as flat as possible to maximize light transmittance perpendicular to the lens plane therethrough. Further, the transition between the flat lens optic portion 1619 and surrounding lens optic portion 1612 can be smoothed to avoid a discontinuous transition (e.g. curve smoothing shape).

In the embodiment shown in FIG. 29, the flat lens optic portions 1619, 1619 are provided on both sides of the lens optic portion 1612. It is noted that the one flat lens optic portion 1619 overlaps the other flat lens optic portion 1619. Alternatively, the flat lens portions can be configured to partially overlap, or not overlap.

Further, it is noted that the one flat lens optic portion 1619 is the same shape (e.g. circle or circular) and the same size (e.g. 1 mm) as the other flat lens portion 1619. Alternatively, the flat lens portion can have a different shape (e.g. oval, triangle, square, rectangle, pentagon, hexagon, octagon, star-shaped, gear-shaped), and/or can have a different size (e.g. overlapping larger circle on one side of lens and small circle on opposite side).

The lens plate haptic portion 1614 is provide with two through holes 1621 to allow anchoring of the accommodating intraocular lens 1610 within the eye.

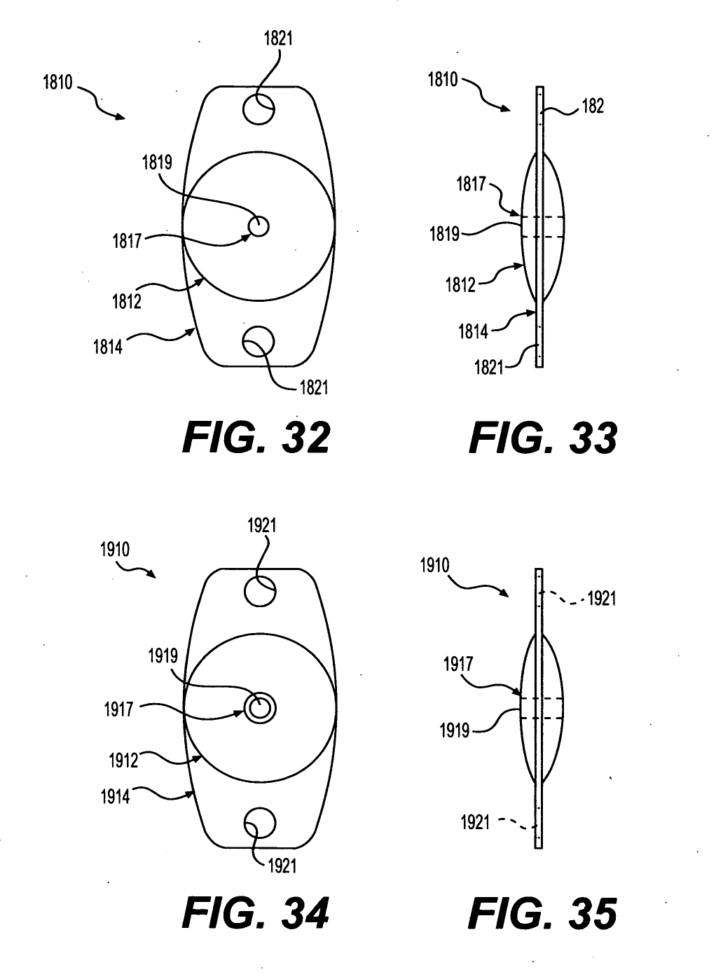

A preferred embodiment of a deformable accommodating intraocular lens device 1810 is shown in FIGS. 32 and 33. The deformable accommodating intraocular lens 1810 provides for static accommodation of vision of the eye.

The deformable accommodating intraocular lens 1810 comprises or consists of a lens optic portion 1812, a plate lens haptic portion 1814, and a lens optic window 1817. In this embodiment, the lens optic window 1817 is configured as a lens optic hole 1819 (e.g. through hole or partial hole or holes) provided on at least one side of the lens optic portion 1812.

The lens optic hole 1819 is located in the lens optic portion 1812. The lens optic hole 1819 is preferably configured to maximize light transmittance perpendicular to the lens plane through the lens optic portion 1812. Further, the transition between the lens optic hole 1819 and surrounding lens optic portion 1812 can be smoothed to avoid a discontinuous transition (e.g. curve smoothing shape).

In the embodiment shown in FIG. 33, the raised flat lens optic portions 1819, 1819 are provided on both sides of the lens optic portion 1812.

A preferred embodiment of a deformable accommodating intraocular lens device 1710 is shown in FIGS. 30 and 31. The deformable accommodating intraocular lens 1710 provides for static accommodation of vision of the eye.

A preferred embodiment of a deformable accommodating intraocular lens device 1910 is shown in FIGS. 34 and 35. The deformable accommodating intraocular lens 1910 provides for static accommodation of vision of the eye.

The deformable accommodating intraocular lens 1910 comprises or consists of a lens optic portion 1912, a plate lens haptic portion 1914, and a lens optic window 1917. In this embodiment, the lens optical window 1917 is configured as a tapering lens optic hole 1919 (e.g. through hole or partial hole or holes) provided on at least one side of the lens optic portion 1912.

The lens optical hole 1919 is located in the lens optic portion 1912. The lens optic hole 1919 is preferably configured to maximize light transmittance perpendicular to the lens plane through the lens optic portion 1912. Further, the transition between the lens optic hole 1919 and surrounding lens optic portion 1912 can be smoothed to avoid a discontinuous transition (e.g. curve smoothing shape).

In the embodiment shown in FIG. 35, the raised flat lens optic portions 1919, 1919 are provided on both sides of the lens optic portion 1912.

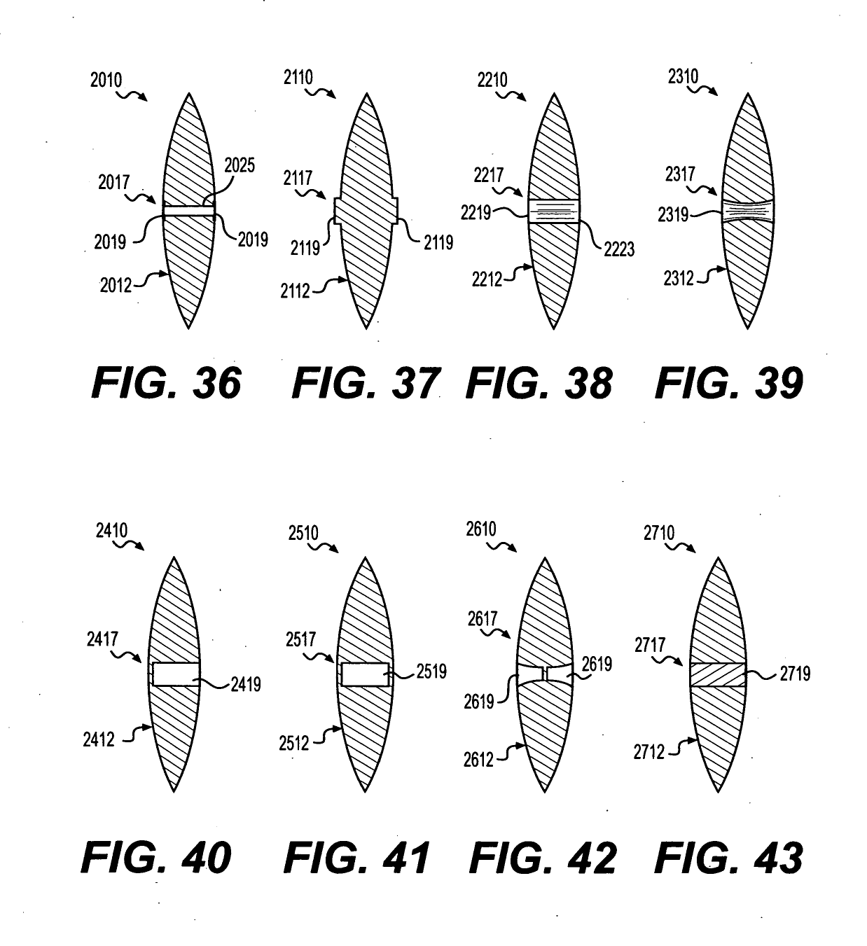

A plurality of embodiments of the accommodating lens device comprising or consisting of a lens optic configured with a lens optic window, is shown in FIGS. 36-51. The lens device embodiments are shown without a haptic or haptics; however, the lens device embodiment can including a haptic or haptics.

An accommodating intraocular lens 2010 comprising of or consisting of a lens optic 2012 is shown in FIG. 36. The lens optic 2012 comprises a lens optic window 2017 configured as at least one flat lens optic portion 2019.

An accommodating intraocular lens 2110 comprising or consisting of a lens optic 2112 is shown in FIG. 37. The lens optic 2112 comprises a lens optic window 2117 configured as at least one raised flat lens optic portion 2119.

An accommodating intraocular lens 2210 comprising or consisting of a lens optic 2212 is shown in FIG. 38. The lens optic 2212 comprises a lens optic window 2217 configured as a lens optic tunnel 2219. The lens optic tunnel is provided with a light barrier 2223 (e.g. frosted surface or insert, opaque surface or insert, colored surface or insert, reflecting surface or insert) to prevent light from the lens optic 2212 from entering the lens optic tunnel 2219.

An accommodating intraocular lens 2310 comprising of or consisting of a lens optic 2312 shown in FIG. 39. The lens optic 2312 comprises a lens optic window 2317 configured as a tapering lens optic hole 2319 tapering outwardly from a center of the lens 2310.

An accommodating intraocular lens 2410 comprising of or consisting of a lens optic 2412 shown in FIG. 40. The lens optic 2412 comprises a lens optic window 2417 configured as a partial depth lens optic hole 2419.

An accommodating intraocular lens 2510 comprising of or consisting of a lens optic 2512 shown in FIG. 41. The lens optic 2512 comprises a lens optic window 2517 configured as a closed ended lens optic hole or cavity 2519.

An accommodating intraocular lens 2610 comprising of or consisting of a lens optic 2612 shown in FIG. 42. The lens optic 2612 comprises a lens optic window 2617 configured as a pair of outwardly tapering lens optic holes 2619.

An accommodating intraocular lens 2710 comprising of or consisting of a lens optic 2712 is shown in FIG. 43. The lens optic 2712 comprises a lens optic window 2717 configured as a lens optic insert 2719 (e.g. material having different refractive index, fiber optic, fiber optic bundle, light guide).

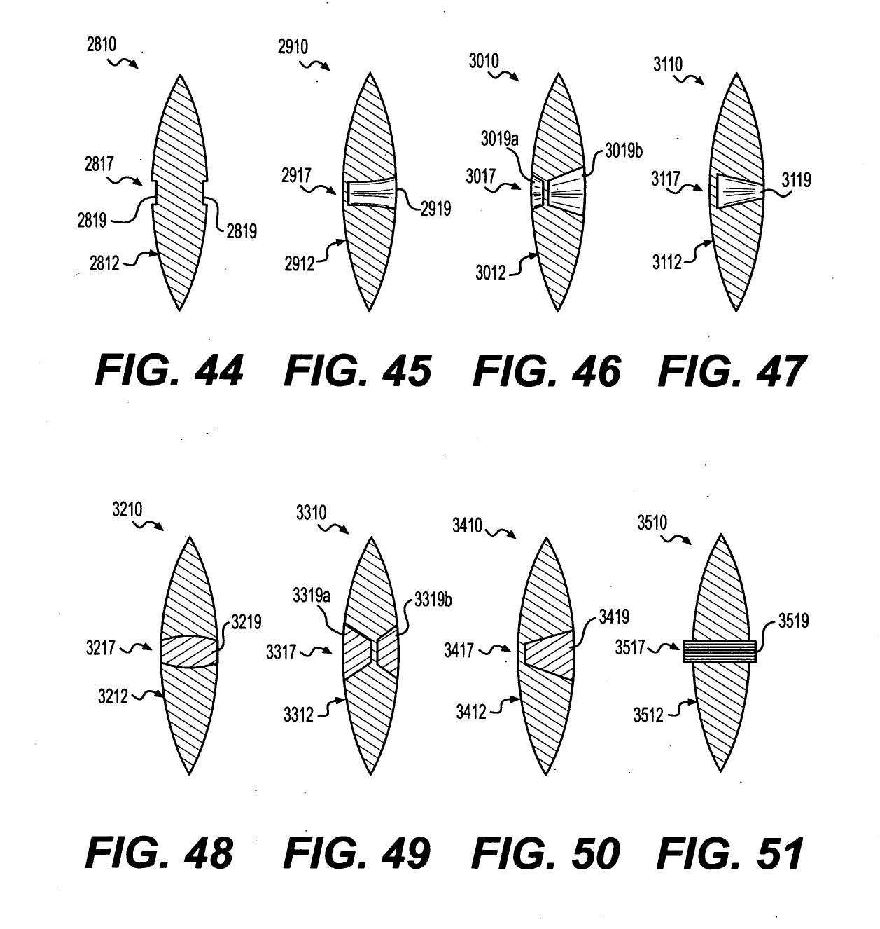

An accommodating intraocular lens 2810 comprising of or consisting of a lens optic 2812 is shown in FIG. 44. The lens optic 2812 comprises a lens optic window 2817 configured as a pair of partial holes or recesses 2819.

An accommodating intraocular lens 2910 comprising of or consisting of a lens optic 2912 is shown in FIG. 45. The lens optic 2912 comprises a lens optic window 2917 configured as an outwardly tapering hole 2919.

An accommodating intraocular lens 3010 comprising of or consisting of a lens optic 3012 is shown in FIG. 46. The lens optic 3012 comprises a lens optic window 3017 configured as a pair of outwardly tapering partial holes or recesses 3019a, 3019b.

An accommodating intraocular lens 3110 comprising of or consisting of a lens optic 3112 is shown in FIG. 47. The lens optic 3112 comprises a lens optic window 3117 configured as an inwardly taping hole 3119.

An accommodating intraocular lens 3210 comprising of or consisting of a lens optic 3212 is shown in FIG. 48. The lens optic 3212 comprises a lens optic window 3217 configured as an elliptical-shaped lens optic insert 3219.

An accommodating intraocular lens 3310 comprising of or consisting of a lens optic 3312 is shown in FIG. 49. The lens optic 3312 comprises or consists of a lens optic window 3317 configured as a pair of outwardly tapering lens optic inserts 3319a, 3319b.

An accommodating intraocular lens 3410 comprising or consisting of a lens optic 3412 is shown in FIG. 50. The lens optic 3412 comprises a lens optic window 3417 configured as an outwardly tapering lens optic insert 2019.

An accommodating intraocular lens 3510 comprising of or consisting of a lens optic 3512 is shown in FIG. 51. The lens optic 3512 comprises a lens optic window 3517 configured as lens optic insert 3519 having outwardly extending ends (e.g. fiber optic bundle, light guide).

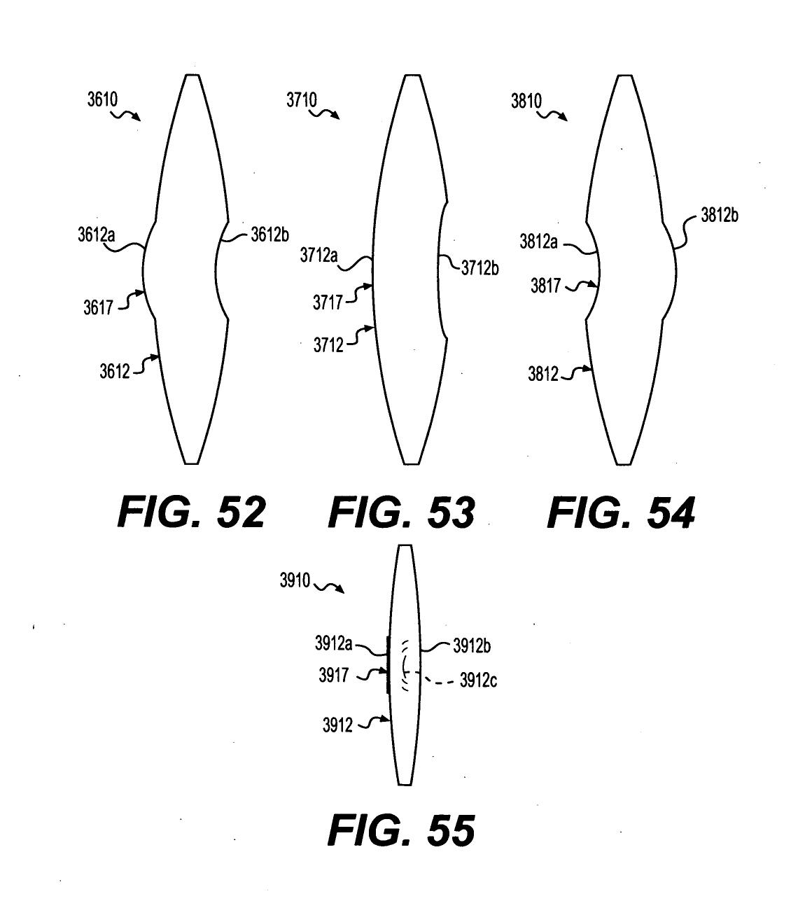

An accommodating intraocular lens 3610 comprising of or consisting of a lens optic 3612 is shown in FIG. 52. The lens optic 3612 comprises a lens optic window 3617 comprising of or consisting of a front positive lens optic surface 3612a and a back negative lens optic surface 3612b.

An accommodating intraocular lens 3710 comprising of or consisting of a lens optic 3712 is shown in FIG. 53. The lens optic 3712 comprises a lens optic window 3717 comprising of or consisting of a front positive lens optic surface 3712a (same as or similar to the surface curvature of the front lens surface) and a back negative lens optic surface 3712b.

An accommodating intraocular lens 3810 comprising of or consisting of a lens optic 3812 is shown in FIG. 54. The lens optic 3812 comprises a lens optic window 3817 comprising of or consisting of a front negative lens optic surface 3812a and a back positive lens optic surface 3612b.

An accommodating intraocular lens 3910 comprising of or consisting of a lens optic 3912 shown in FIG. 54. The lens optic 3912 comprises a lens optic window 3917 comprising of or consisting of a front flat lens optic surface 3812a and a back flat lens optic surface 3812b with a diffractive element 3912c (e.g. diffractive grating).

### Lens Operation

The accommodating intraocular lens is configured to provide accommodation of vision of the eye. The lens optic portion of the intraocular lens, depending on the particular embodiment, can remain fixed in the eye while providing accommodation (e.g. flat lens optic portion embodiment, lens optic light tunnel (e.g. hole or recess) in lens optic embodiment), or can move within the eye to provide accommodation (e.g. lens optic portion connected by flexible arm to plate lens haptic portion), or combination of “static” or fixed type accommodation and “dynamic” or movement type accommodation to provide enhanced accommodation.

In the fixed type accommodation, once implanted, the lens optic portion remains fixed relative to the inside structure of the eye during accommodation operation thereof.

In the movement type accommodation, the accommodating intraocular lens deforms, flexes and/or bows due to forces (e.g. compression, tension, and/or pressure or pressure differential) applied by the eye onto the accommodating intraocular lens (e.g. in particular forces applied to edge portions of the plate haptic portion and/or front and back surfaces of lens).

The accommodating intraocular lens can be located in the anterior chamber and/or posterior chamber of the eye. For example, the intraocular lens can be implanted in: 1) the anterior chamber between the cornea and iris; 2) the posterior chamber between the iris and natural lens; or 3) the posterior chamber in the capsular bag after natural lens removal.

In operation, the accommodating intraocular lens implanted in the capsular bag, for example, are subject to forces that are exerted on the capsular bag by the zonules of the eye. These forces are transmitted to the accommodating intraocular lens, in particular to the peripheral edges thereof. Further, pressure (e.g. differential pressure) can be applied by the eye to the front and back surfaces of the capsular bag and/or the accommodating intraocular lens implanted into the capsular bag. As forces and/or pressures are applied to the accommodating intraocular lens, the plate lens haptic portion begins to deform, flex, or bow in an arch generally perpendicular to the flexible or resilient arm portions connecting the lens optic portion to the plate lens haptic portion so as to move the lens optic portion either forward or rearward from a resting position depending on the particular configuration and arrangement. In any event, the accommodating intraocular lens is configured so that the plate lens haptic portion moves the lens portion during operation.

The operation or functioning of the accommodating intraocular lens 10 is shown in FIGS. 23-26.

The accommodating intraocular lens 10 is shown in an unstressed and unbowed condition, as shown in FIGS. 23 and 24. When inwardly directed forces Fᵢ are applied around the perimeter of the plate lens haptic portion 14, the plate lens haptic portion 14 begins to bow relative to the X axis as illustrated in FIG. 25, and may furthermore be enhanced by varying hydraulic pressures (e.g. differential forces on front and back surfaces of IOL) in the eye. In this manner, the lens optic portion 12 is moved from a resting position in plane P_{R} to an accommodating plane P_{A} and traverses a distance Δ_{A}. However, as illustrated in FIG. 26, the plate lens haptic portion 14 does not bow or substantially bow along the transverse axis Y, since the flexible or resilient arm portion 16, 16 reinforce and stiffen the plate lens haptic portion 14 from bowing along this transverse axis Y. Thus, the accommodating intraocular lens 10 essentially bows in only a single dimension, and not in two (2) dimensions.

As illustrated in FIG. 25, the outer edges of the lens optic portion 12 become somewhat separated and exposed from the upper surface of the plate lens haptic portion 14 due to bowing of the plate lens haptic portion 14. In this manner, it is possible that the lens optic portion 12 could potentially extend into or through the pupil of the iris of the eye. However, as illustrated in FIG. 26, the arm portions 16 do not allow the plate lens haptic portion 14 to bow in the transverse axis Y, and prevents the lens optic portion 12 from being exposed and separate from the upper surface of the plate lens haptic portion 14. Further, the arm portion 16 acts or functions like a pair of bumpers against the back of the iris at the opening of the pupil to prevent the lens optic portion 12 from entering into or passing through the pupil of the iris of the eye.

I claim:

1. An accommodating intraocular lens device configured to be implanted into an eye, the lens device comprising:
a lens optic comprising a lens optic light window configured to provide accommodation of vision of the eye.

2. A lens device according to claim 1, wherein the lens optic light window is configured to allow light rays to pass straight through the lens optic light window parallel to an optical axis of the eye and minimize any light impedance and distortion through the light window.

3. A lens device according to claim 1, wherein the lens optic is a lens optic portion, and further comprising a lens haptic portion connected to the lens optic portion.

4. A lens device according to claim 1, wherein the light window comprises a lens optic light tunnel provided in the lens optic portion.

5. A lens device according to claim 1, wherein the light window comprises a lens optic insert provided in the lens optic portion.

6. A lens device according to claim 1, wherein the lens optic light tunnel is a hole provided in the lens optic portion.

7. The device according to claim 1, wherein lens optic window comprises at least one flat lens optic surface located on at least one side of the lens optic portion.

8. The device according to claim 1, wherein the lens optic portion comprises two flat lens optic surfaces located on opposite sides of the lens optic portion.

9. The device according to claim 8, wherein the two flat lens optic surfaces at least partially overlap each other.

10. The device according to claim 9, wherein the two flat lens optic surfaces are the same size and shape surfaces.

11. The device according to claim 8, wherein the two flat lens optic surfaces are flat circular lens optic surfaces.

12. The device according to claim 11, wherein the circular flat optical surfaces are centered on the lens optic portion.

13. The device according to claim 1, wherein the light window is provided with a light barrier configured to at least partially isolate light passing through the light window from light passing through a surrounding portion of the lens optic portion.

14. The device according to claim 13, wherein the light barrier is an opaque portion of the lens optic portion.

15. The device according to claim 13, wherein the light barrier is formed within the material of the lens optic portion.

16. The device according to claim 13, wherein the light barrier is a separate structure in the lens optic portion.

17. The device according to claim 4, wherein the lens optic light tunnel partially extends through a thickness of the lens optic portion.

18. The device according to claim 4, wherein the lens optic light tunnel fully extends through the thickness of the lens optic portion.

19. The device according to claim 4, wherein at least a portion of an inner surface of the lens optic light tunnel comprises a light barrier surface.

20. The device according to claim 1, wherein said lens window comprises a flat lens optic portion and a light tunnel in the flat lens optic portion.

21. The device according to claim 5, wherein the light tunnel comprises a fiber optic.

22. The device according to claim 6, wherein the lens optic insert is made of a material different from that of the material of the lens optic.

23. The device according to claim 1, wherein the lens optic light window has a width in the range of 0.1 mm to 3.0 mm.

24. A lens device according to claim 1, wherein the light window comprises a front positive lens optic surface and a back negative lens optic surface.

25. A lens device according to claim 1, wherein the light window comprises a front negative lens optic surface and a back positive lens optic surface.

26. A lens device according to claim 1, wherein the light window comprises a diffractive lens optic portion.
